(19) United States
(12) Patent Application Publication
(10) Pub. No.: US 2024/0206802 A1
(43) Pub. Date: Jun. 27, 2024

(54) POSTURE STABILITY DETECTION SYSTEM AND DETECTION METHOD USING THE SAME

(71) Applicant: INDUSTRIAL TECHNOLOGY RESEARCH INSTITUTE, Hsinchu (TW)

(72) Inventors: Hui-Yu CHO, Taichung City (TW); Jian-Hong WU, Caotun Township (TW); Ren-Der JEAN, Hsinchu City (TW); Yao-Hua HO, Taipei City (TW); I-Hung LIAO, Kaohsiung City (TW); Yu-Hsun LEE, Taipei City (TW); Hua-Cheng WENG, New Taipei City (TW)

(73) Assignee: INDUSTRIAL TECHNOLOGY RESEARCH INSTITUTE, Hsinchu (TW)

(21) Appl. No.: 18/143,873

(22) Filed: May 5, 2023

(30) Foreign Application Priority Data
Dec. 21, 2022 (TW) 111149178

(51) Int. Cl. A61B 5/00; A61B 5/103; G06T 7/00

(52) U.S. Cl. CPC A61B 5/4561; A61B 5/0077; A61B 5/1036; G06T 7/0012; A61B 5/742; A61B 2562/0247; A61B 2562/046; G06T 2207/30012

(57) ABSTRACT

A posture stability detection system including a pressure pad, a camera and a processor is provided. The pressure pad is configured to sense a pressure distribution exerted by a to-be-tested subject. The camera is configured to capture a plurality of posture images of the to-be-tested subject. The processor is configured to: obtain a COP position of the to-be-tested subject according to the pressure distribution in a sample; obtain a COP distribution circle of the to-be-tested subject according to the COP positions in several samples; obtain a COP stability value of the to-be-tested subject according to the COP distribution circles; analyze each posture image to obtain a backbone stability value; and, obtain a posture stability value according to the backbone stability value and the COP stability value.

This application claims the benefit of Taiwan application Serial No. 111149178, filed Dec. 21, 2022, the disclosure of which is incorporated by reference herein in its entirety.

### TECHNICAL FIELD

The disclosure relates in general to a posture stability detection system and a stability detection method using the same.

### BACKGROUND

In recent years, along with the popularity of various types of sports home coaching, telerehabilitation therapy and interactive game platform, it has gradually become a trend to detect the user's posture for live video education or feedback interaction or provide the user's posture to the coaches or rehabilitation therapist for posture judgement. To avoid injury, ensure safety and achieve efficiency in sports, the correctness and stability of movement are crucial. Currently, the information of posture provided by the interactive imaging technology mainly focuses on the correctness of movement, and detection involves the use of depth camera, six-axis inertial sensor or pressure sensing device. However, since these devices only could detect the correctness of movement and are unable to determine the stability of movement, sometimes sports injuries occur. Therefore, the core of the technology of the present disclosure is to provide an application related to a system and a detection method using the same related to the stability of movement.

### SUMMARY

The present disclosure provides a posture stability detection system and a method using the same.

According to one embodiment, a posture stability detection system is provided. The posture stability detection system includes a pressure pad, a camera and a processor. The pressure pad is configured to sense a pressure distribution exerted by a to-be-tested subject. The camera is configured to capture a plurality of posture images of the to-be-tested subject. The processor is configured to: obtain a center-of-pressure (COP) position of the to-be-tested subject according to the pressure distribution in a sample; obtain a COP distribution circle of the to-be-tested subject according to the COP positions in several samples; obtain a COP stability value of the to-be-tested subject according to the COP distribution circles; analyze each posture image to obtain a backbone stability value; and, obtain a posture stability value according to the backbone stability value and the COP stability value.

According to another embodiment, a posture stability detection method is provided. The posture stability detection method includes the following steps: sensing a pressure distribution exerted by a to-be-tested subject; capturing a plurality of posture images of the to-be-tested subject; obtain a COP position of the to-be-tested subject according to the pressure distribution in a sample; obtain a COP distribution circle of the to-be-tested subject according to the COP positions in several samples; obtaining a COP stability value of the to-be-tested subject according to the COP distribution circles; analyzing each posture image to obtain a backbone stability value; and, obtaining a posture stability value according to the backbone stability value and the COP stability value.

The above and other aspects of the disclosure will become better understood with regard to the following detailed description of the preferred but non-limiting embodiment (s). The following description is made with reference to the accompanying drawings.

### BRIEF DESCRIPTION OF THE DRAWINGS

FIG. 1 is a functional block diagram of a posture stability detection system according to an embodiment of the present disclosure.

FIG. 2A is a schematic diagram of a pressure pad of a posture stability detection system.

FIG. 2B is a schematic diagram of a to-be-tested subject exerting a pressure on the pressure pad of FIG. 2A.

FIG. 3 is a schematic diagram of a COP distribution circle obtained by the posture stability detection system of FIG. 1.

FIG. 4 is a schematic diagram of a posture image according to an embodiment of the present disclosure.

FIG. 5 is a schematic diagram of a relationship curve of backbone stability value vs time according to an embodiment of the present disclosure.

FIG. 6 is a flowchart of obtaining the COP stability value using the posture stability detection system of FIG. 1.

FIG. 7 is a flowchart of obtaining the backbone stability value using the posture stability detection system of FIG. 1.

### DETAILED DESCRIPTION

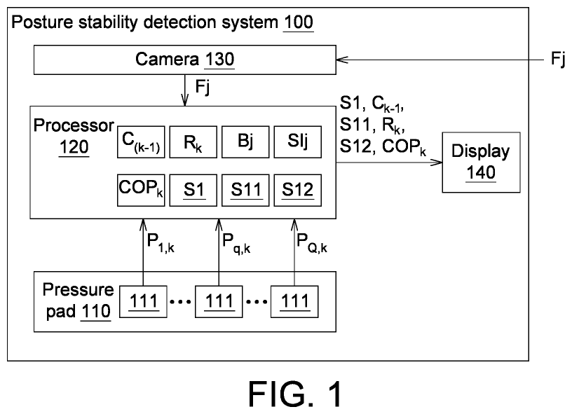

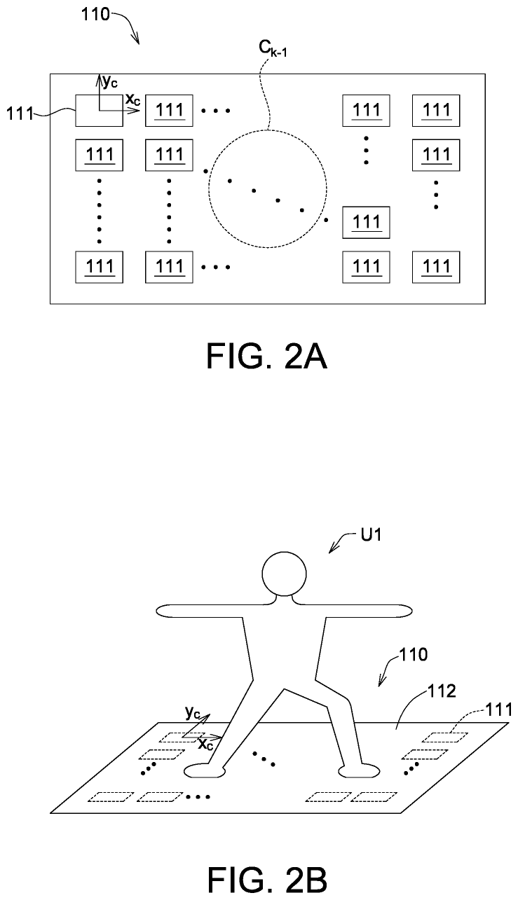

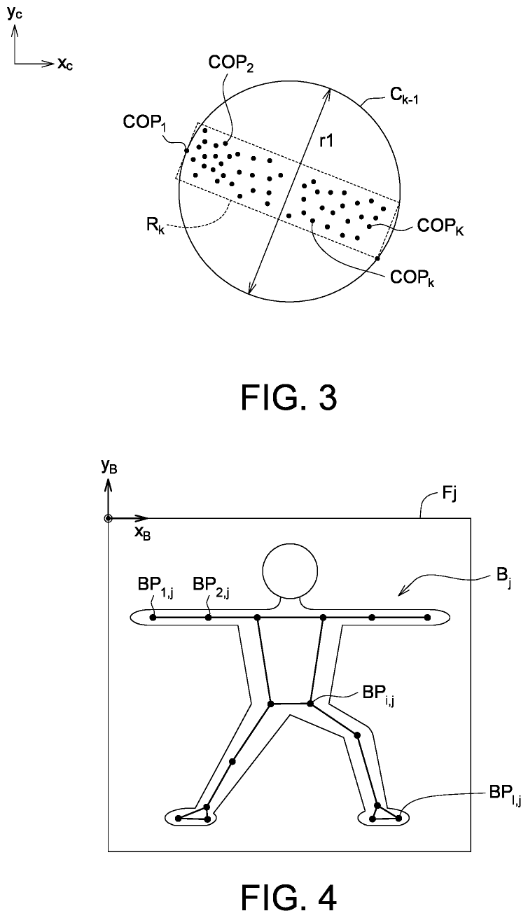

Refer to FIGS. 1 to 4. FIG. 1 is a functional block diagram of a posture stability detection system 100 according to an embodiment of the present disclosure. FIG. 2A is a schematic diagram of a pressure pad 110 of a posture stability detection system 100. FIG. 2B is a schematic diagram of a to-be-tested subject U1 exerting a pressure on the pressure pad of FIG. 2A. FIG. 3 is a schematic diagram of a COP distribution circle C₍ₖ₋₁₎ obtained by the posture stability detection system 100 of FIG. 1. FIG. 4 is a schematic diagram of a posture image Fⱼ according to an embodiment of the present disclosure.

As indicated in FIGS. 1 to 4, the posture stability detection system 100 includes a pressure pad 110, a processor 120, a camera 130 and a display 140. The pressure pad 110 is configured to sense a pressure distribution exerted by a to-be-tested subject U1. The camera 130 is configured to capture J posture images of the to-be-tested subject, wherein J is a positive integer greater than or equivalent to 2. The processor 120 is configured to: (1) obtain a COP position COPₖ of the to-be-tested subject U1 according to the pressure distribution in a sample; (2) obtain a COP distribution circle C₍ₖ₋₁₎ of the to-be-tested subject U1 according to the COP positions in several samples Pₖ; (3) obtain a COP stability value S11 of the to-be-tested subject U1 according to the COP distribution circles C₍ₖ₋₁₎; (4) analyze each posture image Fⱼ to obtain a backbone stability value S12; and, (5) obtain a posture stability value S1 according to the backbone stability value S12 and the COP stability value S11. Thus, through pressure sensing and image analysis, the posture stability detection system 100 could automatically analyze the posture of the to-be-tested subject U1 to obtain a posture stability of the to-be-tested subject U1.

In an embodiment, the pressure pad 110 could be realized by a yoga mat. The processor 120 could be realized by a physical circuit formed by a semiconductor process.

The calculation of the COP stability value S11 is disclosed below.

As indicated in FIGS. 1 and 2A, the pressure pad 110 includes a plurality of pressure sensors 111, wherein each pressure sensor 111 is configured to sense a pressure value P_{q,k} exerted on the pressure pad 110 by a to-be-tested subject. The pressure value P_{q,k} is the pressure value sensed by the q-th pressure sensor 111 at the k-th sample. Depending on the state of pressure exerted by the to-be-tested subject U1 (posture), the pressure values P_{q,k} of two of the pressure sensors 111 could be identical, substantially identical or different. The value of k could be a positive integer between 1 to K. Along with the progress of time, k could be progressively increased (in terms of algorithm or programming, the value of k could be expressed as: k=k+1), the present disclosure does not restrict the value of K. For instance, the value of K could be a positive integer equivalent to or greater than 2, such as 100, 1000, 10000, or sampling times within a period of sampling time, which could be several seconds, several minutes or several hours. In an embodiment, the sampling frequency could be one or more than 1 times per second, but the present disclosure is not limited thereto. Besides, the present disclosure does not specify sampling time, which could be several seconds, several minutes or several hours. Moreover, q is a positive integer between 1 to Q, wherein Q is the number of pressure sensors 111.

In an embodiment as indicated in FIG. 2B, the pressure pad 110 includes plurality of pressure sensors 111 and a carrier 112, wherein pressure sensor 111 could be embedded inside the carrier 112. In another embodiment, the pressure sensor 111 could be attached to the outer surface of the carrier 112. The carrier 112 could be formed of such as rubber, plastics or a combination thereof. In an embodiment, the processor 120 and the pressure pad 110 could be separated from each other. In another embodiment, the processor 120 could be integrated into the pressure pad 110. For instance, the processor 120 could be arranged inside the carrier 112 or on the outer surface thereof. Besides, the processor 120 and the pressure sensors 111 could communicate with each other wirelessly or through a transmission cable connecting the processor 120 and the pressure sensors 111.

As indicated in FIGS. 1 and 2A, the processor 120 is configured to: (1) obtain a COP position COPₖ of a to-be-tested subject according to the pressure value P_{q,k} in a sample; (2) obtain a COP distribution circle C₍ₖ₋₁₎ of the to-be-tested subject according to the COP positions COPₖ in several samples; and, (3) obtain a COP stability value S11 of the to-be-tested subject U1 according to the COP distribution circles C₍ₖ₋₁₎.

For instance, when k=1, the first COP position COPₖ could be generated at the first time of sampling (that is, the k-th sampling). When k=2, the second COP position COPₖ could be generated at the second time of sampling (that is, the k-th) and the first (that is, the (k−1)-th) COP distribution circle C₍ₖ₋₁₎ could be obtained according to the 2 COP positions COPₖ generated at the 2 times of sampling. Then, as indicated in FIG. 3, a COP position COPₖ could be generated at each subsequent times of sampling (the value of k is progressively increased), and the COP distribution circle C₍ₖ₋₁₎ vary with the newly obtained COP position COPₖ. For instance, at the tenth (k=10) sampling, a new COP distribution circle is generated according to all COP positions generated at the 10 times of sampling. To summarize, at the k-th sampling, a new (k−1)-th COP distribution circle C₍ₖ₋₁₎ is generated according to all COP positions generated at the k times of sampling. Additionally, the first radius r1 of the COP distribution circle C₍ₖ₋₁₎ varies with the distribution of the COP position COPₖ generated after the most updated sampling. Moreover, at the k-th sampling, the processor 120 obtains the COP distribution circle C₍ₖ₋₁₎ according to all COP positions COPₖ covered by the COP distribution circle C₍ₖ₋₁₎ and the smallest radius, wherein at least one COP position COPₖ could be located on the circumference of the COP distribution circle C₍ₖ₋₁₎.

As indicated in FIGS. 1 and 2A, the COP position COPₖ is the COP position at the k-th sampling. The COP position COPₖ could be represented by the coordinates of the COP position (xCOPₖ, yCOPₖ). The coordinates of the COP position (xCOPₖ, yCOPₖ) could be obtained with reference to the x_{C}-y_{C} coordinate system, wherein xCOPₖ represents the x_{C} axis coordinate value of the COP position COPₖ and yCOPₖ represents the y_{C} axis coordinate value of the COP position COPₖ. The original point of the x_{C}-y_{C} coordinate system could be set to any position of the pressure pad 110, such as any pressure sensor 111 on the pressure pad 110. Each pressure sensor 111 has a set of sensor coordinates (x_{q}, y_{q}), and the pressure sensors 111 at different positions have different sensor coordinates (x_{q}, y_{q}), wherein the subscript q represents the q-th pressure sensor 111.

In an embodiment, the COP position COPₖ at each time of sampling could be obtained according to the following formulae (1A) to (1B).

$${xCOP}_{k} = \frac{{\sum }_{q = 1}^{Q} {x}_{q} \cdot {P}_{q , k}}{{\sum }_{q = 1}^{Q} {P}_{q , k}} \qquad ( 1 A )$$

$${yCOP}_{k} = \frac{{\sum }_{q = 1}^{Q} {y}_{q} \cdot {P}_{q , k}}{{\sum }_{q = 1}^{Q} {P}_{q , k}} \qquad ( 1 B )$$

After the COP distribution circle C₍ₖ₋₁₎ is obtained, a COP stability value S11 could be obtained according to the COP distribution circle C₍ₖ₋₁₎. One of the calculation methods of the COP stability value S11 is disclosed below.

As indicated in formulae (2A) to (2E), the processor 120 is further configured to: (1) obtain a first area A1 corresponding to a first radius r1 of the COP distribution circle C₍ₖ₋₁₎ according to formula (2A); (2) obtain a second area A2 corresponding to a second radius r2 according to formula (2B); (3) obtain a square root squ of an area difference (ΔA=A1−A2) between the first area A1 and the second area A2 according to formula (2C); (4) obtain a product value sca of the square root squ and a radius ratio ra according to formula (2D), wherein the radius ratio ra is equivalent to a ratio (r1/r2) of the first radius r1 to the second radius r2; and, (5) obtain a difference value between 100 and the product value sca according to formula (2E), and use the difference value as the COP stability value S11.

$$A 1 = r {1}^{2} \cdot π \qquad ( 2 A )$$

$$A 2 = r {2}^{2} \cdot π \qquad ( 2 B )$$

$$squ = \sqrt{( A 1 - A 2 )} \qquad ( 2 C )$$

$$sca = squ \cdot ( \frac{r 1}{r 2} ) \qquad ( 2 D )$$

$$S 11 = 100 - s c a \qquad ( 2 E )$$

In formulae (2A) to (2B), the first radius r1 is the radius value of the COP distribution circle C₍ₖ₋₁₎ generated after each time of sampling, and the second radius r2 is the radius value of the COP distribution circle of a reference posture. The first radius r1 is not smaller than the second radius r2. The smaller the difference between the COP stability value S11 of the to-be-tested subject and the COP stability value of the reference posture, the smaller the difference value between the first radius r1 and the second radius r2, and the larger the COP stability value S11 (the maximum is 100). The larger the difference between the COP stability value S11 of the to-be-tested subject and the COP stability value of the reference posture, the larger the difference value between the first radius r1 and the second radius r2 and the smaller the COP stability value S11.

In an embodiment, the reference posture is the standard posture of a person demonstrating standard posture (for instance, a yoga teacher), and the calculation of the COP distribution circle could be identical or similar to the calculation of the COP distribution circle C₍ₖ₋₁₎. In the present example, the COP stability value S11 is the stability value of the to-be-tested subject U1 relative to the standard posture. In another embodiment, the reference posture could be the posture of the to-be-tested subject U1. For instance, the second radius r2 could be the radius value of one of the COP distribution circles (the number is k−1) of C₍ₖ₋₁₎ of the to-be-tested subject U1 generated at previous k times of sampling, an average of radius values or a function of several radius values of the (k−1) COP distribution circles C₍ₖ₋₁₎ of the to-be-tested subject U1 generated at previous k times of sampling. In the present example, the COP stability value S11 is the stability value of the to-be-tested subject U1 relative to past posture of the same to-be-tested subject U1. In other embodiments, the radius value of the COP distribution circle of the reference posture could be a predetermined value. Moreover, the COP distribution circles of the reference posture generated at any two times of sampling could be identical or different.

Besides, the calculation of the COP stability value S11 is not limited to the method exemplified in the embodiment of the present disclosure and could also be obtained using other methods.

As indicated in FIG. 3, after each time of sampling, the processor 120 is configured to: cover all COP positions COPₖ with an inscribed rectangle Rₖ. The left-and-right stability of the body of the to-be-tested subject U1 could be observed through the inscribed rectangle Rₖ. For instance, the posture stability whether both hands and both feet of the to-be-tested subject U1 press the pressure pad 110 could be observed. For instance, the inscribed rectangle Rₖ could present whether the pressure exerted by the left-hand side of the body (left hand and left foot) and the pressure exerted by the right-hand side of the body (right hand and right foot) are balanced or whether the pressure exerted by one hand or one foot is too heavy. If yes, the inscribed rectangle Rₖ will not be parallel to the horizontal axis. Through the observation of the inscribed rectangle Rₖ, the to-be-tested subject U1 could adjust the exerted pressure and determine whether the movement requiring overall balance meets the standard or not.

As indicated in FIGS. 1 and 3, the display 140 is electrically connected to the processor 120. The processor 120 could transmit the information of COP stability (for instance, the COP position COPₖ, the COP distribution circle C₍ₖ₋₁₎ and/or inscribed rectangle Rₖ) of the to-be-tested subject U1 generated at each time of sampling to the display 140 for the display 140 to display. The to-be-tested subject U1 could obtain their own COP stability through the information displayed on the display 140. In an embodiment, the processor 120 could transmit the information of COP stability (for instance, the COP position COPₖ, the COP distribution circle C₍ₖ₋₁₎ and/or inscribed rectangle Rₖ) of the reference posture demonstrator generated at each time of sampling to the display 140 for the display 140 to display. Thus, the display 140 could concurrently display the information of COP stability of the to-be-tested subject U1 and the information of COP stability of the reference posture demonstrator; through the displayed information, the to-be-tested subject could obtain the difference between their own COP stability of the to-be-tested subject and that of the reference posture demonstrator. Apart from displaying the information of COP stability, the display 140 further could display the scope of the pressure pad 110, so that the to-be-tested subject U1 could obtain the information of COP stability relative to the position/region of the pressure pad 110.

The calculation of the backbone stability value S12 is disclosed below.

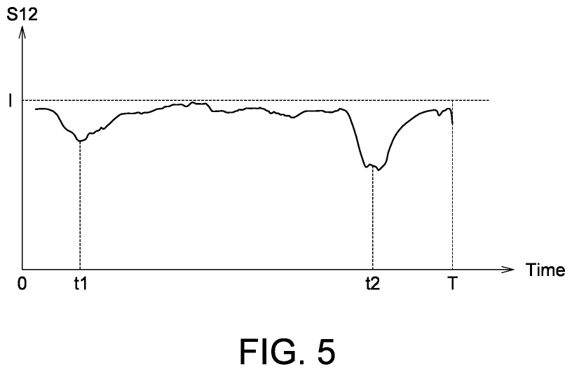

Refer to FIGS. 4 and 5. FIG. 5 is a schematic diagram of a relationship curve of backbone stability value S12 vs time according to an embodiment of the present disclosure.

As indicated in FIGS. 1 and 4, the camera 130 is configured to capture J posture images Fⱼ of the to-be-tested subject U1, wherein the subscript j is a positive integer between 1 to J, and J represents the number of posture images Fⱼ, such as the total number of frames within a period of time, which could be several seconds, several minutes, or several hours. The present disclosure does not specify the value of J, and the value of J could be determined according to the frame rate (frames/second) and the capture sampling time of the camera 130. The processor 120 further is configured to: (1) analyze each posture image Fⱼ to obtain a backbone Bⱼ of each posture image Fⱼ; and, (2) obtain a backbone stability value S12 according to the changes in J backbones Bⱼ of the J posture images Fⱼ.

As indicated in FIG. 4, the backbone Bⱼ of the j-th posture image Fⱼ includes at least one feature point BP_{i,j}, which represents the i-th feature point on the j-th posture image Fⱼ, wherein the value of I could be between 1 to I, and I represents the total number of feature points BP_{i,j}, such as 33 or could be larger or smaller than 33. The embodiment of the present disclosure does not restrict the value of I. The feature point BP_{i,j} could be the joint point and/or the rotation pivot of the backbone Bⱼ. Each feature point BP_{i,j} could be represented by feature coordinates (xᵢ^{F}^{ⱼ}, yᵢ^{F}^{ⱼ}), wherein xᵢ^{F}^{ⱼ} represents the x axis coordinate value of the i-th feature point BP_{i,j} on the j-th posture image Fⱼ and yᵢ^{F}^{ⱼ} represents the y axis coordinate value of the i-th feature point BP_{i,j} on the j-th posture image Fⱼ. The feature coordinates (xᵢ^{F}^{ⱼ}, yᵢ^{F}^{ⱼ}) could be created with reference to x_{B}-y_{B} coordinate system, and the original point of the x_{B}-y_{B} coordinate system could be created at any position of the posture image Fⱼ, such as a corner of the posture image Fⱼ. The position of the original point of the x_{B}-y_{B} coordinate system is the same for each posture image Fⱼ. The feature coordinates (xᵢ^{F}^{ⱼ}, yᵢ^{F}^{ⱼ}) of each feature point BP_{i,j} on the same posture image Fⱼ are different. Depending on the posture of the to-be-tested subject U1, the values of the feature coordinate (xᵢ^{F}^{ⱼ}, yᵢ^{F}^{ⱼ}) of the same feature point BP_{i,j} on each posture image Fⱼ could be identical or different.

The processor 120 further is configured to: (1) obtain the average coordinates (xᵢ^{avg}, yᵢ^{avg}) of the i-th feature coordinates (xᵢ^{F}^{ⱼ}, yᵢ^{F}^{ⱼ}) on J posture images Fⱼ; (2) calculate cosine similarity between the I average coordinates (xᵢ^{avg}, yᵢ^{avg}) and the I feature coordinates (xᵢ^{F}^{ⱼ}, yᵢ^{F}^{ⱼ}) of the j-th posture image Fⱼ to obtain a cosine similarity value SIⱼ, and use the cosine similarity value SIⱼ as the backbone stability value S12.

The said average coordinates (xᵢ^{avg}, yᵢ^{avg}) could be obtained according to formulae (3A) to (3B).

$${x}_{i}^{a v g} = \frac{{\sum }_{j = 1}^{J} {x}_{i}^{{F}_{j}}}{J} \qquad ( 3 A )$$

$${y}_{i}^{a ν g} = \frac{{\sum }_{j = 1}^{J} {y}_{i}^{{F}_{j}}}{J} \qquad ( 3 B )$$

xᵢ^{avg} represents the x axis average coordinate value of the i-th feature point BP_{i,j} on J posture images Fⱼ. Take the first feature point BP_{1,j} for instance. The average coordinate x₁^{avg} of the first feature point BP_{i,j} could be obtained by dividing the sum of the x axis coordinate value x₁^{F}^{ⱼ} of the first feature point BP_{1,j} on each posture image Fⱼ by J. The average coordinate xᵢ^{avg} of each of the remaining (i=2 to I) feature points BP_{i,j} could be obtained by the same analogy, and the similarities are not repeated here. Similarly, yᵢ^{avg} represents the y axis average coordinate value of the i-th feature point BP_{i,j} on J posture images Fⱼ. Take the first feature point BP_{1,j} for instance. The average coordinate y₁^{avg} of the first feature point BP_{1,j} could be obtained by dividing the sum of the y axis coordinate value y₁^{F}^{ⱼ} of the first feature point BP_{1,j} on each posture image Fⱼ. The average coordinate yᵢ^{avg} of each of the remaining (i=2 to I) feature points BP_{i,j} could be obtained by the same analogy, and the similarities are not repeated here. For the i-th feature point on J posture images Fⱼ, there is only one set of x axis average value xᵢ^{avg} and y axis average value yᵢ^{avg}, which is represented by average coordinates (xᵢ^{avg}, yᵢ^{avg}).

After the average coordinates (xᵢ^{avg}, yᵢ^{avg}) are obtained, the j-th cosine similarity value SIⱼ could be obtained according to formulae (4A) to (4C).

$${SI}_{j} = similarity ( \vec{A} , \vec{B} ) = \frac{\vec{{A}_{J}} \cdot \vec{{B}_{J}}}{‖ \vec{{A}_{J}} ‖ * ‖ \vec{{B}_{J}} ‖} \qquad ( 4 A )$$

$$\vec{{A}_{J}} = [ {x}_{1}^{avg} , {y}_{1}^{avg} , … , {x}_{i}^{avg} , {y}_{i}^{avg} , … , {x}_{I}^{avg} , {y}_{I}^{a v g} ] \qquad ( 4 B )$$

$$\vec{{B}_{J}} = [ {x}_{1}^{{F}_{j}} , {y}_{1}^{{F}_{j}} , … , {x}_{i}^{{F}_{j}} , {y}_{i}^{{F}_{j}} , … , {x}_{I}^{{F}_{j}} , {y}_{I}^{{F}_{j}} ] \qquad ( 4 C )$$

In formula (4B), vector Aⱼ represents the set of average coordinates (xᵢ^{avg}, yᵢ^{avg}) of I feature points BP_{i,j} on the j-th posture image Fⱼ, and the dimension is 2I. For instance, when there are 33 feature points (I=33), the dimension of vector Aⱼ is 66. In formula (4C), vector Bⱼ represents the set of feature coordinates (xᵢ^{F}^{ⱼ}, yᵢ^{F}^{ⱼ}) of I feature points BP_{i,j} on the j-th posture image Fⱼ, and the dimension is 2I. In formula (4A), the processor 120 calculates cosine similarity between vector Aⱼ and vector Bⱼ to obtain a cosine similarity SIⱼ of the j-th posture image Fⱼ. Furthermore, a cosine similarity SIⱼ could be generated from a posture image Fⱼ, and several cosine similarities SIⱼ could be generated from several posture images Fⱼ to construct a cosine similarity curve. The cosine similarity SIⱼ could be a result of comparison between the backbone stability of the j-th posture image Fⱼ of the to-be-tested subject per se and the backbone average stability. The smaller the difference between the backbone stability of the j-th posture image Fⱼ of the to-be-tested subject per se and the backbone average stability, the smaller the difference value between the cosine similarity SIⱼ and 1. Conversely, the larger the difference between the backbone stability of the j-th posture image Fⱼ of the to-be-tested subject per se and the backbone average stability, the larger the between difference value the cosine similarity SIⱼ and 1. Thus, the to-be-tested subject U1 could obtain their own backbone stability through the changes in the cosine similarity SIⱼ.

Also, after a period of time, the processor 120 could obtain J backbone stability values S12 within the period of time. As indicated in FIG. 5, depending on the posture of the to-be-tested subject U1, the J backbone stability values S12 may vary with the time. At time points t1 and t2, the backbone stability value S12 deviates from 1, and such deviation indicates relatively unstable. The backbone stability value S12 at the remaining time points being relatively close to 1 than at time point t1 and t2 indicates relative stability.

As indicated in FIGS. 1 and 5, the processor 120 transmits the backbone stability value S12 of the to-be-tested subject U1 to the display 140 for the display 140 to display, so that the to-be-tested subject U1 could obtain the changes in their backbone stability through the backbone stability value S12 displayed on the display 140. Apart from displaying the backbone stability value S12 of the to-be-tested subject U1, the display 140 could further display the backbone stability value of the one demonstrating standard posture for the to-be-tested subject U1 to obtain the difference between their own backbone stability value S12 and the backbone stability value of the one demonstrating standard posture.

The calculation of the posture stability value S1 is disclosed below.

In an embodiment, the processor 120 could obtain a posture stability value S1 according to the COP stability value S11 and the backbone stability value S12. For instance, the processor 120 is configured to obtain the posture stability value S1 according to the formula (5) below.

$$S 1 = a \times S 11 + b \times S 12 \qquad ( 5 )$$

In formula (5), the values of parameters a and b are not fixed and could be adjusted according to the movements of the to-be-tested subject U1. In an embodiment, the sum of a and b is equivalent to 1, but the present disclosure is not limited thereto. In another embodiment, the posture stability value S1 could omit one of the COP stability value S11 and the backbone stability value S12.

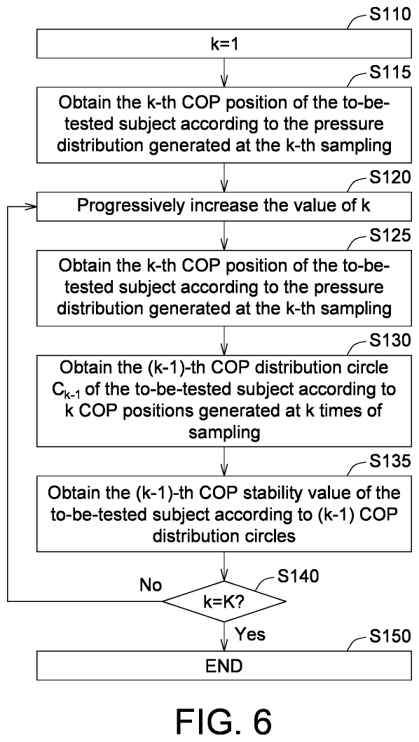

Referring to FIG. 6, a flowchart of obtaining the COP stability value S11 using the posture stability detection system of FIG. 1 is shown.

In step S110, the initial value of k is set to 1 by the processor 120.

In step S115, the first COP position COP₁ of the to-be-tested subject (the first COP position COP₁ is illustrated in FIG. 3) is obtained by the processor 120 according to the pressure distribution generated at the k-th sampling (the first time of sampling). The first COP position COP₁ could be obtained according to the above formulae (1A) to (1B), and the similarities are not repeated here.

In step S120, the value of k is progressively increased by the processor 120.

In step S125, the k-th COP position COPₖ of the to-be-tested subject (the k-th COP position COPₖ is illustrated in FIG. 3) is obtained by the processor 120 according to the pressure distribution generated at the k-th sampling. The k-th COP position COPₖ could be obtained according to the above formulae (1A) to (1B), and the similarities are not repeated here.

In step S130, the (k−1)-th COP distribution circle Cₖ₋₁ of the to-be-tested subject (the COP distribution circle Cₖ₋₁ is illustrated in FIG. 3) is obtained by the processor 120 according to k COP positions COPₖ generated at k times of sampling.

In step S135, the (k−1)-th COP stability value S11 of the to-be-tested subject is obtained by the processor 120 according to (k−1) COP distribution circles Cₖ₋₁. The COP stability value S11 could be obtained according to the above formulae (2A) to (2E), and the similarities are not repeated here.

In step S140, whether the value of k is equivalent to K is determined by the processor 120. If yes, the process proceeds to step S150, and the calculation of the COP stability value S11 terminates. If no, the process returns to step S120, the processor 120 progressively increases the value of k and continues to obtain the COP stability value S11 at the next sampling.

Other steps of obtaining the COP stability value by the posture stability detection system 100 are already disclosed above, and the similarities are not repeated here.

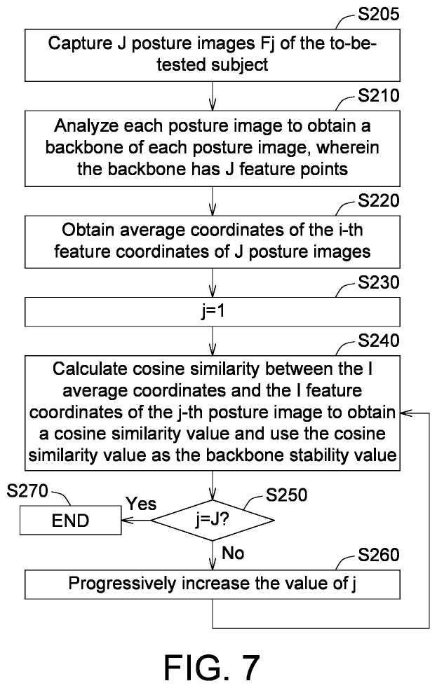

Referring to FIG. 7, a flowchart of obtaining the backbone stability value S12 using the posture stability detection system 100 of FIG. 1 is shown.

In step S205, J posture images Fⱼ of the to-be-tested subject U1 within a period of time are captured by the camera 130, wherein the period of time could be several seconds or minutes.

In step S210, each posture image Fj is analyzed by the processor 120 to obtain a backbone Bj of each posture image Fj. The backbone Bⱼ includes at least one feature point BP_{i,j}, and the processor 120 could analyze backbone Bj by using image analysis technology to obtain the feature coordinates (xᵢ^{F}^{ⱼ}, yᵢ^{F}^{ⱼ}) of the feature points BP_{i,j} of the backbone Bⱼ.

In step S220, average coordinates (xᵢ^{avg}, yᵢ^{avg}) of the i-th feature coordinates (xᵢ^{F}^{ⱼ}, yᵢ^{F}^{ⱼ}) of J posture images F_{j could} be obtained by the processor 120 according to formulae (3A) to (3B). If the number of feature coordinates (xᵢ^{F}^{ⱼ}, yᵢ^{F}^{ⱼ}) of the feature point BP_{i,j} is I, the processor 120 could obtain I average coordinates (xᵢ^{avg}, yᵢ^{avg}).

In step S230, the initial value of j is set to 1 by the processor 120.

In step S240, the j-th cosine similarity value SIⱼ is obtained by the processor 120 according to formulae (4A) to (4C). Cosine similarity between the I average coordinates (xᵢ^{avg}, yᵢ^{avg}) and the I feature coordinate (xᵢ^{F}^{ⱼ}, yᵢ^{F}^{ⱼ}) of the j-th posture image Fⱼ is calculated to generate a cosine similarity value SIⱼ, and the cosine similarity value SIⱼ is used as the backbone stability value S12.

In step S250, whether the value of j has reached J is determined by the processor 120. If yes, the process proceeds to step S270, the calculation of the backbone stability value S12 terminates. If no, the process proceeds to step S260, the processor 120 progressively increases the value of j (in terms of algorithm or programming, the value of j is expressed as j=j+1), then the process returns to step S240, the processor 120 continues to obtain the backbone stability value S12 of the next posture image Fⱼ.

Other steps of calculating the COP stability value by the posture stability detection system 100 are disclosed above, and the similarities are not repeated here.

After obtaining the COP stability value S11 and the backbone stability value S12, the processor 120 could obtain the posture stability value S1 according to formula (5).

To summarize, the present disclosure provides a posture stability detection system and a posture stability detection method using the same. Through pressure sensing and/or image analysis, the posture stability detection system could automatically analyze the posture of a to-be-tested subject to obtain the posture stability of the to-be-tested subject.

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A posture stability detection system, comprising:
a pressure pad configured to sense a pressure distribution exerted by a to-be-tested subject;
a camera configured to capture a plurality of posture images of the to-be-tested subject; and
a processor configured to:
obtain a center-of-pressure (COP) position of the to-be-tested subject according to the pressure distribution in a sample;
obtain a COP distribution circle of the to-be-tested subject according to a plurality of the COP positions in a plurality of samples;
obtain a COP stability value of the to-be-tested subject according to a plurality of the COP distribution circles;
analyze each posture image to obtain a backbone stability value; and
obtain a posture stability value according to the backbone stability value and the COP stability value.

2. The posture stability detection system according to claim 1, wherein the pressure pad comprises a plurality of pressure sensors, and each pressure sensor is configured to:
sense a pressure value exerted on the pressure pad by the to-be-tested subject;
the processor further is configured to:
obtain the COP position of the to-be-tested subject according to the pressure values in the sample; and
obtain the COP distribution circle of the to-be-tested subject according to the COP positions in the samples;
obtain the COP stability value of the to-be-tested subject according to a plurality of the COP distribution circles.

3. The posture stability detection system according to claim 2, wherein the processor further is configured to:
obtain a first area corresponding to a first radius of one of the COP distribution circles;
obtain a second area corresponding to a second radius;
obtain a square root of an area difference between the first area and the second area;
obtain a product value of the square root and a radius ratio, wherein the radius ratio is equivalent to a ratio of the first radius to the second radius; and
obtain a difference value between 100 and the product value and use the difference value as the COP stability value.

4. The posture stability detection system according to claim 1, wherein the processor further is configured to:
analyze each posture image to obtain a backbone of each posture image; and
obtain the backbone stability value according to the changes in the backbones of the posture images.

5. The posture stability detection system according to claim 4, wherein the backbone of each posture image comprises a feature point having a feature coordinate value; the processor further is configured to:
obtain an average coordinate of the feature coordinates of the posture images; and
calculate a cosine similarity between the average coordinate and each feature coordinate of the posture images to generate a cosine similarity value and use the cosine similarity value as the backbone stability value.

6. The posture stability detection system according to claim 1, wherein the processor further is configured to cover all of the COP positions with an inscribed rectangle.

7. The posture stability detection system according to claim 1, further comprising:
a display electrically connected to the processor and configured to:
display at least one of the COP distribution circle, the COP stability value and the backbone stability value.

8. The posture stability detection system according to claim 1, further comprising:
a display electrically connected to the processor and configured to:
display the COP distribution circle of the to-be-tested subject and the COP distribution circle of a standard posture.

9. A posture stability detection method, comprising:
sensing a pressure distribution exerted by a to-be-tested subject;
capturing a plurality of posture images of the to-be-tested subject;
obtaining a COP position of the to-be-tested subject according to the pressure distribution in a sample;
obtaining a COP distribution circle of the to-be-tested subject according to a plurality of the COP positions in a plurality of samples;
obtaining a COP stability value of the to-be-tested subject according to a plurality of the COP distribution circles;
analyzing each posture image to obtain a backbone stability value; and
obtaining a posture stability value according to the backbone stability value and the COP stability value.

10. The posture stability detection method according to claim 9, further comprising:
sensing a pressure value exerted on the pressure pad by the to-be-tested subject;
obtaining the COP position of the to-be-tested subject according to the pressure values in the sample; and
obtaining the COP distribution circle of the to-be-tested subject according to the COP positions in the samples;
obtaining the COP stability value of the to-be-tested subject according to a plurality of the COP distribution circles.

11. The posture stability detection method according to claim 10, further comprising:
obtaining a first area corresponding to a first radius of one of the COP distribution circles;
obtaining a second area corresponding to a second radius;
obtaining a square root of an area difference between the first area and the second area;
obtaining a product value of the square root and a radius ratio, wherein the radius ratio is equivalent to a ratio of the first radius to the second radius; and
obtaining a difference value between 100 and the product value and using the difference value as the COP stability value.

12. The posture stability detection method according to claim 9, further comprising:
analyzing a backbone of each posture image to obtain each posture image; and
obtaining the backbone stability value according to the changes in the backbones of the posture images.

13. The posture stability detection method according to claim 12, wherein the backbone of each posture image comprises a feature point having a feature coordinate value; the posture stability detection method further comprises:
obtaining an average coordinate of the feature coordinates of the posture images; and
calculating a cosine similarity between the average coordinate and each feature coordinate of the posture images to generate a cosine similarity value and using the cosine similarity value as the backbone stability value.

14. The posture stability detection method according to claim 9, further comprising:
covering all of the COP positions with an inscribed rectangle.

15. The posture stability detection method according to claim 9, further comprising:
displaying at least one of the COP distribution circle, the COP stability value and the backbone stability value.

16. The posture stability detection method according to claim 9, further comprising:
displaying the COP distribution circle of the to-be-tested subject and the COP distribution circle of a standard posture.
